(19) United States
(12) Patent Application Publication
(10) Pub. No.: US 2012/0058470 A1
(43) Pub. Date: Mar. 8, 2012

(54) ELECTRICAL WIRING OF POLYNUCLEOTIDES FOR NANOELECTRONIC APPLICATIONS

(75) Inventors: Nickolas Chelyapov, Los Angeles, CA (US); Mohamed Y. El-Naggar, Claremont, CA (US)

(73) Assignee: UNIVERSITY OF SOUTHERN CALIFORNIA, Los Angeles, CA (US)

(21) Appl. No.: 13/320,244

(22) PCT Filed: May 13, 2010
(86) PCT No.: PCT/US10/34819

§ 371 Date: Nov. 11, 2011

Related U.S. Application Data
(60) Provisional application No. 61/177,896, filed on May 13, 2009.

(51) Int. Cl. C12Q 1/68; G01N 33/566

(52) U.S. Cl. 435/6.1; 436/501

(57) ABSTRACT

The present invention relates to incorporation and patterning of polynucleotide molecular wires onto surfaces. In one embodiment, two or more thiol-modified polynucleotide anchors are separately attached to metal contacts that are in turn separately attached to a substrate. Each polynucleotide anchor contains an unpaired region of bases that when bound to complimentary regions of a polynucleotide bridge molecule allow for electrical communication between contacts, and therefore detection of the polynucleotide bridge.

### CROSS-REFERENCE TO RELATED APPLICATIONS

This application claims priority from U.S. Provisional Patent Application No. 61/177,896, filed on May 13, 2009, which is incorporated herein by reference in its entirety.

### FIELD OF THE INVENTION

The invention generally relates to the incorporation and patterning of polynucleotide molecular wires onto surfaces.

### BACKGROUND

The following description includes information that may be useful in understanding the present invention. It is not an admission that any of the information provided herein is prior art or relevant to the presently claimed invention, or that any publication specifically or implicitly referenced is prior art.

Polynucleotides are an attractive candidate for molecular wires in nanoelectronic devices due to its unique properties of self-assembly, based on complementary recognition, and relative chemical stability (1). The electrical properties of polynucleotides, however, are far less understood, with various research groups reporting behaviors ranging from insulating to semiconductive to superconductive (2). The wide disparity in experimental results is likely due to the measurement conditions, namely the environment (solution, dry, coupling to substrate, substrate type, etc.), length, nucleotide sequence of the polynucleotide molecules, and the means by which polynucleotides are coupled to the measurement electrodes. A methodology for robust wiring of polynucleotides, single and/or double-stranded, of arbitrary lengths and sequences, in electronic devices that can be functional under various environmental conditions, is highly desirable. This motivates the current invention, where the inventors describe a framework for utilizing the unique assembly and recognition properties of polynucleotides to self-wire metallic sites on micro/nano electronic chips.

### SUMMARY OF THE INVENTION

In one embodiment, the invention includes a system, comprising: a substrate; a first contact attached to the substrate; a second contact attached to the substrate; a first polynucleotide attached to the first contact and having a binding region; a second polynucleotide attached to the second contact and having a binding region; and a bridging polynucleotide having a first binding region attached to the binding region of the first polynucleotide and a second binding region attached to the binding region of the second polynucleotide. In another embodiment, the system further comprises one or more electronic components in electrical communication with the substrate and/or the first contact and/or the second contact. In some embodiments the substrate comprises silicon or glass. In some embodiments, the first and second contacts each comprise a material independently selected from the group consisting of: aluminum, antimony, arsenic, barium, beryllium, bismuth, boron, cadmium, cesium, chromium, cobalt, copper, gallium, germanium, gold, hafnium, indium, iron, lead, lithium, manganese, mercury, molybdenum, nickel, platinum, palladium, rhodium, iridium, osmium, ruthenium, rhenium, rubidium, scandium, selenium, silver, strontium, tantalum tellurium, thallium, thorium, tin, titanium, tungsten, vanadium, zinc, zirconium and combinations thereof. In some embodiments, the 5′ end of each of the first and second polynucleotides are thiol-modified and covalently attached to the first and second contacts respectively. In certain embodiments, the attachment of the first and second binding regions of the bridging polynucleotide to the binding regions of the first and second polynucleotides, respectively, is achieved via hybridization of complementary base pairs.

In some embodiments, the invention further comprises one or more additional contacts attached to the substrate. In certain embodiments, the invention may further comprise one or more additional polynucleotides each attached to one or more of the one or more additional contacts. In some embodiments, the invention further comprises one or more additional bridging polynucleotides each comprising a first binding region and a second binding region, wherein the first binding region of each additional bridging polynucleotide is attached to a binding region of one of the one or more additional polynucleotides, and the second binding region of each additional bridging polynucleotide is attached to a binding region of a different one of the one or more additional polynucleotides. In some embodiments, the invention further comprises one or more molecules attached to the bridging polynucleotide, the one or more molecules independently selected from the group consisting of proteins, drugs, chemical markers, polymerases, nucleases, antibiotics and combinations thereof. In certain embodiments, the attached molecules are adapted to adopt a pattern influenced by a distribution pattern of the first contact, the second contact and the one or more additional contacts on the substrate.

In another embodiment, the invention includes a device, comprising: a substrate; a first contact attached to the substrate; a second contact attached to the substrate; a first polynucleotide attached to the first contact and having a binding region; and a second polynucleotide attached to the second contact and having a binding region. In another embodiment, the device further comprises an electronic component in electrical communication with the substrate and/or the first contact and/or the second contact. In another embodiment, the electronic component is to detect current flowing between the first contact and the second contact. In another embodiment, the substrate comprises silicon. In still another embodiment, the first and second contacts each comprise a material independently selected from the group consisting of: aluminum, antimony, arsenic, barium, beryllium, bismuth, boron, cadmium, cesium, chromium, cobalt, copper, gallium, germanium, gold, hafnium, indium, iron, lead, lithium, manganese, mercury, molybdenum, nickel, platinum, palladium, rhodium, iridium, osmium, ruthenium, rhenium, rubidium, scandium, selenium, silver, strontium, tantalum tellurium, thallium, thorium, tin, titanium, tungsten, vanadium, zinc, zirconium and combinations thereof. In another embodiment of the device, the 5′ end of each of the first and second polynucleotides are thiol-modified and covalently attached to the first and second contacts respectively. In another embodiment, the device further comprises one or more additional contacts attached to the substrate. In another embodiment, the device further comprises one or more additional polynucleotides each attached to one or more of the one or more additional contacts and each having a binding region, wherein either all binding regions are identical or all binding regions are not identical. In another embodiment, the device is configured to interact with a quantity of bridging polynucleotides, each comprising a first binding region and a second binding region configured to individually attach to the binding regions of the one or more additional polynucleotides. In another embodiment of the invention, the first binding region of the quantity of bridging polynucleotides is the same on all bridging polynucleotides. In another embodiment of the invention, the second binding region of the quantity of bridging polynucleotides is the same on all bridging polynucleotides. In another embodiment of the device, the first binding region of the quantity of bridging polynucleotides is not the same on all bridging polynucleotides. In another embodiment of the invention, the second binding region of the quantity of bridging polynucleotides is not the same on all bridging polynucleotides.

In another embodiment, the invention includes a method for detecting polynucleotide sequences, comprising: providing a device, comprising: a substrate, a first contact attached to the substrate, a second contact attached to the substrate, a first polynucleotide attached to the first contact and having a binding region, and a second polynucleotide attached to the second contact and having a binding region; contacting the device with one or more polynucleotide sequences, each with a first and second region of one or more unpaired bases; introducing an electrical current to the device; and testing for conduction of the electrical current between the first and second contacts, wherein electrical conduction detected between the first and second contacts indicates hybridization of one of the one or more polynucleotide sequences to each of the first and second polynucleotides, and a lack of electrical conduction detected between the first and second contacts indicates a lack of hybridization of a polynucleotide sequence to each of the first and second polynucleotides. In another embodiment of the invention, the testing further comprises employing one or more on-chip and/or external devices that are attached to the device. In another embodiment of the invention, one of the one or more on-chip and/or external devices is an ammeter and/or a voltmeter. In yet another embodiment of the invention, the device is configured to measure conductance and/or capacitance. In another embodiment of the invention, the device further comprises one or more additional contacts attached to the substrate, and one or more additional polynucleotides each attached to one or more of the one or more additional contacts and each having a binding region, wherein either all binding regions are identical or all binding regions are not identical, and wherein testing for conduction of the electrical current further comprises testing for conduction of the electrical current between any pair of contacts included among the first contact, the second contact and the one or more additional contacts.

In another embodiment, the invention includes a method for determining single molecule kinetics, comprising: providing a wiring system comprising: a substrate, a first electrode attached to the substrate, a second electrode attached to the substrate, and a polynucleotide attached to the first electrode; applying a positive bias to the second electrode; and monitoring the time elapsed between applying the positive bias to the second electrode and the attachment of the polynucleotide to the second electrode to determine single molecule kinetics.

In another embodiment, the invention includes a method for determining and analyzing the electrical properties of a polynucleotide sequences, comprising: providing a device, comprising: a substrate, a first contact attached to the substrate, a second contact attached to the substrate, a first polynucleotide attached to the first contact and having a binding region, and a second polynucleotide attached to the second contact and having a binding region; contacting the device with a bridging polynucleotide sequence that hybridizes to the binding regions of each of the first and second polynucleotides; and introducing an electrical current to the device; and measuring the electrical properties of the bridging polynucleotide using a device selected from the group consisting of: semiconductor parameter analyzers, voltmeters, ammeters, pulse generators, potentiostats, galvanostats, function generators and combinations thereof, and wherein the measured electrical properties are selected from the group consisting of: conductance, capacitance, inductance and combinations thereof.

Other features and advantages of the invention will become apparent from the following detailed description, taken in conjunction with the accompanying drawings, which illustrate, by way of example, various embodiments of the invention.

### BRIEF DESCRIPTION OF THE DRAWINGS

Exemplary embodiments are illustrated in referenced figures. It is intended that the embodiments and figures disclosed herein are to be considered illustrative rather than restrictive.

FIG. 1 depicts a schematic describing a methodology for electrical wiring of polynucleotides in micro/nano electronic devices in accordance with an embodiment of the present invention.

FIG. 2 depicts directed attachment of a bridging polynucleotide on polynucleotides bound to contacts in accordance with an embodiment of the present invention. (A) Positive bias favors the flow of the free bridging polynucleotide to the left electrode. Once hybridized to the left anchor (B), polarity is switched and the free end of the bridging polynucleotide is directed towards the right electrode/anchor, resulting in a fully wired unit (C).

FIG. 3 depicts a schematic describing a methodology for electrical wiring of polynucleotides in micro/nano electronic devices in accordance with an embodiment of the present invention. The schematic further demonstrates the attachment of the electrical wiring to on-chip electrical components.

FIG. 4 depicts a schematic describing a methodology for electrical wiring of polynucleotides in micro/nano electronic devices in accordance with an embodiment of the present invention. The schematic further demonstrates the attachment of the electrical wiring to external electrical components.

### DETAILED DESCRIPTION OF THE INVENTION

All references cited herein are incorporated by reference in their entirety as though fully set forth. Unless defined otherwise, technical and scientific terms used herein have the same meaning as commonly understood by one of ordinary skill in the art to which this invention belongs. Singleton et al., Dictionary of Microbiology and Molecular Biology 3^{rd} ed., J. Wiley & Sons (New York, N.Y. 2001); March, Advanced Organic Chemistry Reactions, Mechanisms and Structure 5^{th} th ed., J. Wiley & Sons (New York, N.Y. 2001); and Sambrook and Russel, Molecular Cloning: A Laboratory Manual 3rd ed., Cold Spring Harbor Laboratory Press (Cold Spring Harbor, N.Y. 2001), provide one skilled in the art with a general guide to many of the terms used in the present application.

One skilled in the art will recognize many methods and materials similar or equivalent to those described herein, which could be used in the practice of the present invention. Indeed, the present invention is in no way limited to the methods and materials described.

“Conductive contact” as used herein means a conductive material attached to a surface suitable for micro/nanofabrication such as a silicon-based substrate.

“Bridging polynucleotide” as used herein means a single- or double-stranded polynucleotide.

“Sticky end” as used herein means the region of unpaired polynucleotide bases available for hybridization.

A first embodiment of the invention includes a polynucleotide-wired device 1, which may include a substrate 103, at least two contacts 102 configured on the substrate 103, and a quantity of polynucleotides individually attached to one or more of the contacts 100, wherein at least some of the at least two contacts 102 have one polynucleotide 100 from the quantity of polynucleotides 100 bound thereto. The device 1 may further include a quantity of bridging polynucleotides 105, wherein at least some pairs of the polynucleotides 100 that are themselves bound to contacts 102 and that themselves each have a recognition site are also in biochemical communication with one another via a bridging polynucleotide 105 that hybridizes to each of the pair of polynucleotides bound to contacts 102 at their respective recognition sites 100b. In one embodiment of the invention the substrate 103 is silicon-based. In other embodiments, the substrate 103 is glass. In certain embodiments of the invention the contacts 102 are made of metal. In other embodiments, the contacts 102 each comprise a material independently selected from the group consisting of: aluminum, antimony, arsenic, barium, beryllium, bismuth, boron, cadmium, cesium, chromium, cobalt, copper, gallium, germanium, gold, hafnium, indium, iron, lead, lithium, manganese, mercury, molybdenum, nickel, platinum, palladium, rhodium, iridium, osmium, ruthenium, rhenium, rubidium, scandium, selenium, silver, strontium, tantalum tellurium, thallium, thorium, tin, titanium, tungsten, vanadium, zinc, zirconium and combinations thereof. In a particular embodiment of the invention the contacts 102 are made of gold. In some embodiments of the invention the polynucleotides 100 bound to contacts are thiol-modified. In one embodiment of the invention the thiol-modified polynucleotides are each bound to a gold contact, which are in turn each bound to a silicon substrate. In certain embodiments the contacts are in communication with an on-chip detection device 106. In some embodiments the contacts are in communication with an external detection device 107. One of skill in the art will readily appreciate that a wide variety of configurations of the inventive device may be utilized for various purposes, each of said configurations and purposes being contemplated as being within the scope of the present invention.

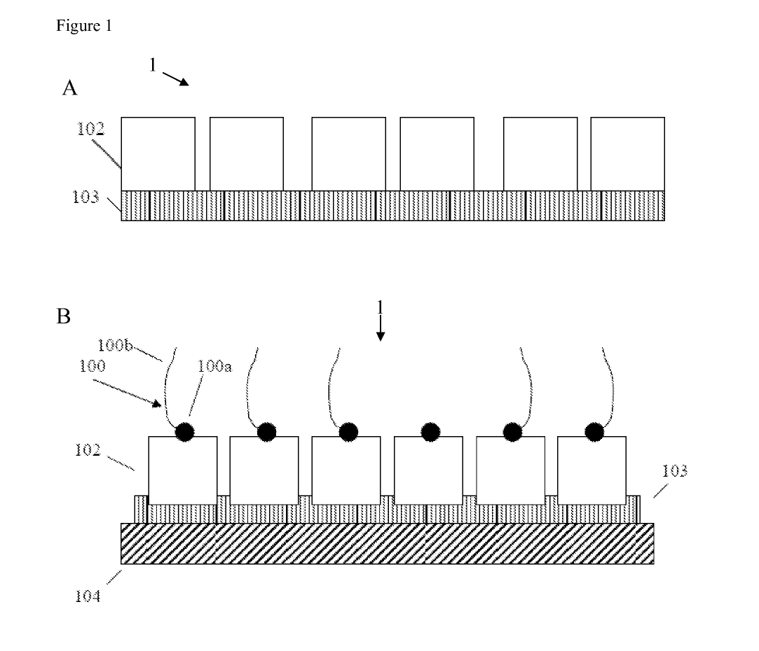

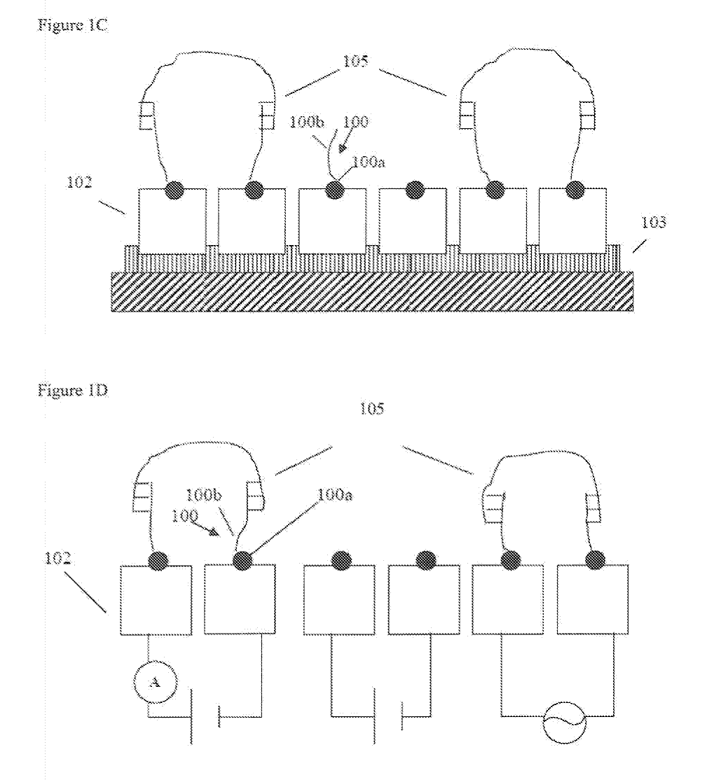

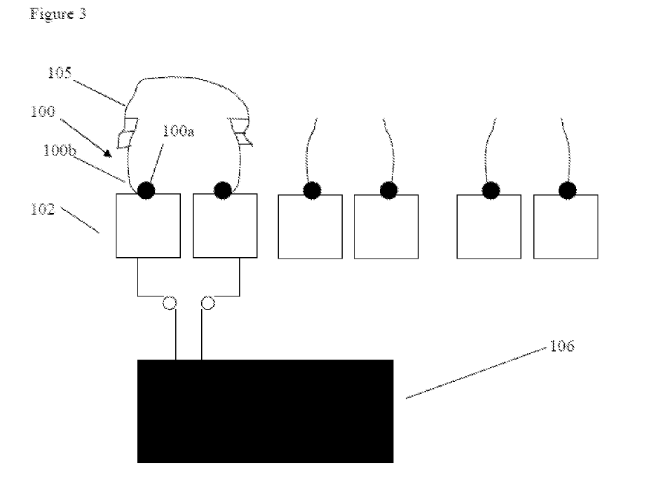

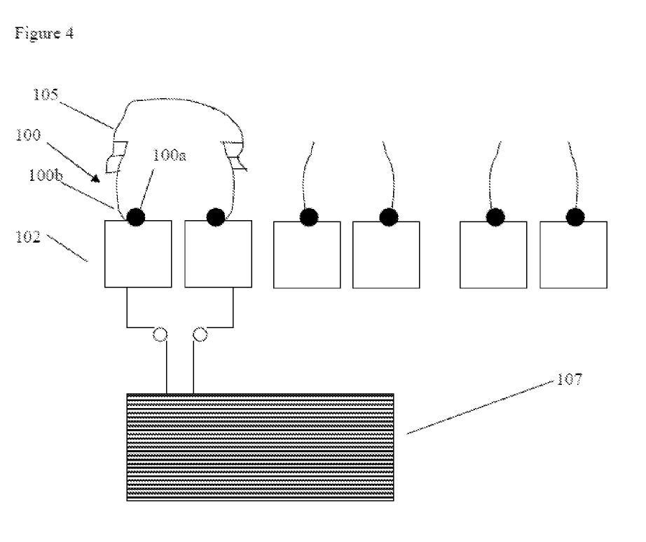

In one embodiment of the present invention, a procedure for preparing electrical wiring of a polynucleotide is schematically illustrated in FIG. 1, starting with a pattern of contacts 102 on a surface suitable for micro/nanofabrication 103 (FIG. 1A). Chemically modified polynucleotide molecules 100 are then attached to the conductive sites 102 (FIG. 1B). These single-stranded or double-stranded polynucleotides 100 bound to contacts 102 have specific recognition sites (sticky ends) at the free ends 100b, which enable them to act as probes for incoming polynucleotide strands 105 (bridging polynucleotides). The latter 105 then self-assemble, to bridge the gap between contacts 102, by hybridizing with complimentary sequences at the free ends 100b of polynucleotide probes 100 on these sites. In one embodiment of the invention, two or more contact sites on the surface are connected upon introduction of a sufficiently complimentary bridging polynucleotide sequence FIG. 1C. In another embodiment of the invention, the polynucleotide-wired circuit prepared as set forth above may be connected to various on-chip 106 or external electronic components 107 (FIG. 3,4). In one embodiment, the on-chip electronic components 106 are used for detection of a specific sequence 105 (FIG. 3). In another embodiment, the external electronic components 107 are used for detection of a specific sequence 105 (FIG. 4). In certain embodiments of the invention, electrical current is applied to on or more contacts. In other embodiments, the flow of current is measured between pairs of contacts bound to polynucleotides. In another embodiment of the invention, combinations of two or more polynucleotides bound to contacts are used to analyze/detect a multitude of bridging polynucleotide sequences. In certain embodiments, the polynucleotides have distinct binding sequences that are available for binding one or more regions of bridging polynucleotides. In other embodiments the binding regions of the polynucleotides bound to the contacts are the same. In certain embodiments two or more bridging polynucleotide sequences are detected simultaneously. In certain embodiments, the sequences are detected sequentially according to the order of detection. In other embodiments, the order of detection is determined by the order in which various contacts are electrically interrogated to determine binding of a bridging polynucleotide. In certain embodiments, probes that measure the flow of electricity are permanently attached to the contacts for measuring current flowing between them. In other embodiments, the probes are temporarily contacted to the contacts in order to detect the flow of current between the contacts. In certain embodiments of the invention, on-chip 106 or external electrical components 107 are used to analyze the electrical properties of the bridging polynucleotide 105. In some embodiments of the invention the bridging polynucleotide 105 is bound to one or more molecules selected from the group consisting of: proteins, chemical markers, drugs and combinations thereof. In certain embodiments, the molecules are further selected from the group consisting of polymerases, nucleases, or antibiotics. In some embodiments of the invention, the respective contributions of various characteristics of bridging polynucleotides to conductance and/or capacitance are determined by step-wise testing. In some embodiments the various characteristics are selected from the group consisting of: secondary structure, length, base modifications, and effects of binding a bridging polynucleotide to other molecules. In certain embodiments of the invention, polynucleotide sequences are designed and wired according to their electronic properties. In some embodiments of the invention, the wiring is used to generate geometries/patterns that form the backbone of a polynucleotide nanoelectronic device. In yet another embodiment, the backbone polynucleotide may be bound in one or more regions to one or more molecules selected from the group comprising: proteins, drugs, chemical markers, and combinations thereof. In certain embodiments, the molecules are further selected from the group consisting of polymerases, nucleases, or antibiotics.

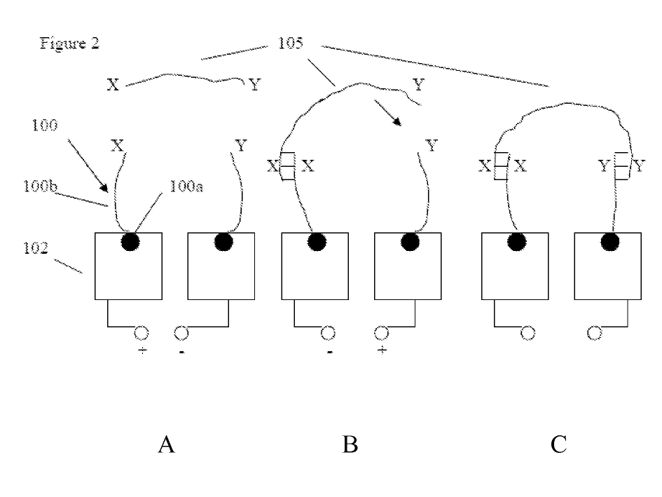

In one embodiment of the invention, specific individual polynucleotide molecules 100 are attached to defined electrodes 102 by applying a positive voltage bias to the desired electrodes, while applying a negative bias to others to prevent attachment to these sites (FIG. 2A). The positive bias then attracts the negatively-charged functionalized polynucleotide molecules 100 and upon contact of the polynucleotide molecule with the contact 102 surface, binding will occur (FIG. 2B). In one embodiment of the invention the functionalized polynucleotide molecules are thiol-modified at an appropriate end to attach to the gold (Au) electrode. In one embodiment of the invention non-bound polynucleotide molecules are washed away. In another embodiment of the invention the procedure may be repeated to create a “forest” of sticky-end-specific polynucleotides 100 attached to individual electrodes 102. In another embodiment, pairs of sticky-end-specific polynucleotides 100 are used to detect specific sequences of bridging polynucleotide sequences 105. In one embodiment, voltage bias is used to attach the incoming polynucleotide bridges onto two or more electrodes 102/100 using a two or more step procedure. In one embodiment, positive bias is applied to the first electrode 102/100 while the counter-electrode 102 receives negative bias (FIG. 2A). The bridging polynucleotide 105 with a sticky end complementary to that of the polynucleotide bound to the positive electrode is then directed towards the positive electrode 102 (FIG. 2A). In some embodiments of the invention, after hybridization resulting from the previous step, the polarity is switched and the free end of the bridging polynucleotide 105 is attracted towards the newly positive electrode 102/100, causing the yet un-hybridized sticky end to bind to the free anchor's sticky end (FIG. 2B). In certain embodiments, hybridization with a positive bias is not used at every stage. In one embodiment, after binding of one end of a bridging polynucleotide 105 to a polynucleotide attached to a contact 100, passive diffusion-based attachment (without bias) is used to attach the other sticky end on a bridging polynucleotide 105 to the other polynucleotide attached to another contact 100.

### EXAMPLES

The following examples are provided to better illustrate the claimed invention and are not to be interpreted as limiting the scope of the invention. To the extent that specific materials are mentioned, it is merely for purposes of illustration and is not intended to limit the invention. One skilled in the art may develop equivalent means or reactants without the exercise of inventive capacity and without departing from the scope of the invention.

### Example 1

### Probing Polynucleotide Electrical Properties

Conductance and capacitance of specific bridging polynucleotide sequences are compared in order to determine the effects of secondary structure, length, and base modifications on polynucleotide electrical properties. Electrical properties tested include conductance (resistance), capacitance and inductance. Circuits are made by combinations of elements useful in this regard (e.g., conductors, capacitors, resistors, etc.). Also all these properties can be investigated in any range of frequencies. Tests of electrical properties are conducted using standard electronic equipment such as semiconductor parameter analyzers, voltmeters, ammeters, pulse generators, potentiostats, galvanostats, function generators and other standard equipment. Bridging polynucleotides of varying lengths, ratios of bases, patterns of bases, and quantities and types of modified bases are generated, and conductance and capacitance are determined by attaching these fragments to polynucleotides attached to contacts linked to detectors. Capacitance, conductance and inductance of numerous samples are compared in order to determine the individual and cumulative effects of the aforementioned variables. Conductance, capacitance and inductance are also tested under various levels of ambient humidity and with varying compositions of contacts and substrates. Contacts are made from aluminum, antimony, arsenic, barium, beryllium, bismuth, boron, cadmium, cesium, chromium, cobalt, copper, gallium, germanium, gold, hafnium, indium, iron, lead, lithium, manganese, mercury, molybdenum, nickel, platinum, palladium, rhodium, iridium, osmium, ruthenium, rhenium, rubidium, scandium, selenium, silver, strontium, tantalum tellurium, thallium, thorium, tin, titanium, tungsten, vanadium, zinc, zirconium and combinations thereof. Further, proteins, chemical markers, drugs and combinations thereof are bound to bridging polynucleotides and tested in the manner previously described, in order to determine the effect on conductance, capacitance and inductance.

### Example 2

### Construction of Polynucleotide-Based Nanoelectronic Devices

A polynucleotide-based transistor is made using metallic electrodes for source, drain, and gate, and a semiconductive polynucleotide wire as the channel. Metallic electrodes are composed of metals including, aluminum, antimony, arsenic, barium, beryllium, bismuth, boron, cadmium, cesium, chromium, cobalt, copper, gallium, germanium, gold, hafnium, indium, iron, lead, lithium, manganese, mercury, molybdenum, nickel, platinum, palladium, rhodium, iridium, osmium, ruthenium, rhenium, rubidium, scandium, selenium, silver, strontium, tantalum tellurium, thallium, thorium, tin, titanium, tungsten, vanadium, zinc, zirconium and combinations thereof. Specific sequences and modifications of polynucleotide wires are chosen for a given location on the transistor, according to the desired electrical properties of the wire.

### Example 3

### Polynucleotide-Based Encryption

Polynucleotide-based encryption is accomplished using the recognition properties of polynucleotides. Specifically, bridging polynucleotide molecules (keys) are introduced and hybridized to polynucleotides bound to contacts at specific locations with sticky ends of known sequences, thereby altering the electrical properties in those locations. Electrically interrogating these locations provides a read-out signal, detecting whether hybridization of a given bridging polynucleotide has occurred, and thereby testing whether the correct key was introduced to obtain the appropriate electrical signal. These locations are be interrogated using electronic probe stations, wire bonding, or even using specially made devices that “sit on” the chips. The readout signal is then communicated to the user by translating it into a result, displayed on a graphical user interface.

### Example 4

### Polynucleotide-Based Templating of Advanced Materials

A desired pattern of advanced materials is created using polynucleotide-based templating. Polynucleotide wires are attached to a substrate in a desired pattern. The polynucleotide is chemically modified (before or after binding to the substrate), allowing for binding of other molecules or particles onto these wires. The molecules bound to the polynucleotide wires assume a pattern that is influenced by the pattern of polynucleotides. The pattern of bound materials is partly dependent upon the pattern of polynucleotides attached to the substrate, and partly dependent upon the nature of the bound materials. In one example, carbon nanotubes are used.

### Example 5

### Single Molecule Kinetic Studies

Starting with a single molecule attached to an electrode/anchor assembly, a positive bias is applied to the counter electrode (FIG. 2B). A measurement is taken of the time elapsed between applying the bias and the successful attachment of the molecule to the counter electrode (FIG. 2C). This measurement yields a readout electrical signal indicating the conductance and/or capacitance measured. The dependence of time on binding events to the polynucleotide molecule is used to study polynucleotide-ligand interactions. Ligands used are proteins, chemical markers, drugs, and combinations thereof. In one example, DNA modifying enzymes, such as polymerases or nucleases are used.

Various embodiments of the invention are described above in the Detailed Description. While these descriptions directly describe the above embodiments, it is understood that those skilled in the art may conceive modifications and/or variations to the specific embodiments shown and described herein. Any such modifications or variations that fall within the purview of this description are intended to be included therein as well. Unless specifically noted, it is the intention of the inventors that the words and phrases in the specification and claims be given the ordinary and accustomed meanings to those of ordinary skill in the applicable art(s).

### References Cited:

1. C. Dekker, M. A. Ratner, Physics World 14, 29 (2001).
2. R. G. Endres, D. L. Cox, R. R. P. Singh, Reviews of Modern Physics 76, 195 (2004).
3. T. P. Beebe, C. E. Rabke-Clemmer (U.S. Pat. No. 5,472,881, 1995)
4. H. Cohen, C. Nogues, R. Naaman, D. Porath, Proceedings of the National Academy of Sciences of the United States of America 102, 11589 (2005).
5. P. Reep (U.S. patent application Ser. No. 11/622,136 2007)
6. See, for example, Integrated DNA Technologies, Inc. (http://www.idtdna.com)

We claim:

1. A system, comprising:
a substrate;
a first contact attached to the substrate;
a second contact attached to the substrate;
a first polynucleotide attached to the first contact and having a binding region;
a second polynucleotide attached to the second contact and having a binding region; and
a bridging polynucleotide having a first binding region attached to the binding region of the first polynucleotide and a second binding region attached to the binding region of the second polynucleotide.

2. The system of claim 1, further comprising one or more electronic components in electrical communication with the substrate and/or the first contact and/or the second contact.

3. The system of claim 1, wherein the substrate comprises silicon or glass.

4. The system of claim 1, wherein the first and second contacts each comprise a material independently selected from the group consisting of: aluminum, antimony, arsenic, barium, beryllium, bismuth, boron, cadmium, cesium, chromium, cobalt, copper, gallium, germanium, gold, hafnium, indium, iron, lead, lithium, manganese, mercury, molybdenum, nickel, platinum, palladium, rhodium, iridium, osmium, ruthenium, rhenium, rubidium, scandium, selenium, silver, strontium, tantalum tellurium, thallium, thorium, tin, titanium, tungsten, vanadium, zinc, zirconium and combinations thereof.

5. The system of claim 1, wherein the 5′ end of each of the first and second polynucleotides are thiol-modified and covalently attached to the first and second contacts respectively.

6. The system of claim 1, wherein the attachment of the first and second binding regions of the bridging polynucleotide to the binding regions of the first and second polynucleotides, respectively, is achieved via hybridization of complementary base pairs.

7. The system of claim 1, further comprising one or more additional contacts attached to the substrate.

8. The system of claim 7, further comprising one or more additional polynucleotides each attached to one or more of the one or more additional contacts.

9. The system of claim 8, further comprising one or more additional bridging polynucleotides each comprising a first binding region and a second binding region,
wherein the first binding region of each additional bridging polynucleotide is attached to a binding region of one of the one or more additional polynucleotides, and the second binding region of each additional bridging polynucleotide is attached to a binding region of a different one of the one or more additional polynucleotides.

10. The system of claim 1, further comprising one or more molecules attached to the bridging polynucleotide, the one or more molecules independently selected from the group consisting of proteins, drugs, chemical markers, polymerases, nucleases, antibiotics and combinations thereof.

11. The system of claim 10, wherein the attached molecules are adapted to adopt a pattern influenced by a distribution pattern of the first contact, the second contact and the one or more additional contacts on the substrate.

12. A device, comprising:
a substrate;
a first contact attached to the substrate;
a second contact attached to the substrate;
a first polynucleotide attached to the first contact and having a binding region; and
a second polynucleotide attached to the second contact and having a binding region.

13. The device of claim 12, further comprising an electronic component in electrical communication with the substrate and/or the first contact and/or the second contact.

14. The device of claim 13, wherein the electronic component is to detect current flowing between the first contact and the second contact.

15. The device of claim 12, wherein the substrate comprises silicon.

16. The device of claim 12, wherein the first and second contacts each comprise a material independently selected from the group consisting of: aluminum, antimony, arsenic, barium, beryllium, bismuth, boron, cadmium, cesium, chromium, cobalt, copper, gallium, germanium, gold, hafnium, indium, iron, lead, lithium, manganese, mercury, molybdenum, nickel, platinum, palladium, rhodium, iridium, osmium, ruthenium, rhenium, rubidium, scandium, selenium, silver, strontium, tantalum tellurium, thallium, thorium, tin, titanium, tungsten, vanadium, zinc, zirconium and combinations thereof.

17. The device of claim 12, wherein the 5′ end of each of the first and second polynucleotides are thiol-modified and covalently attached to the first and second contacts respectively.

18. The device of claim 12, further comprising one or more additional contacts attached to the substrate.

19. The device of claim 13, further comprising one or more additional polynucleotides each attached to one or more of the one or more additional contacts and each having a binding region, wherein either all binding regions are identical or all binding regions are not identical.

20. The device of claim 19, wherein the device is configured to interact with a quantity of bridging polynucleotides, each comprising a first binding region and a second binding region configured to individually attach to the binding regions of the one or more additional polynucleotides.

21. The device of claim 20, wherein the first binding region of the quantity of bridging polynucleotides is the same on all bridging polynucleotides.

22. The device of claim 20, wherein the second binding region of the quantity of bridging polynucleotides is the same on all bridging polynucleotides.

23. The device of claim 20, wherein the first binding region of the quantity of bridging polynucleotides is not the same on all bridging polynucleotides.

24. The device of claim 20, wherein the second binding region of the quantity of bridging polynucleotides is not the same on all bridging polynucleotides.

25. A method for detecting polynucleotide sequences, comprising:
providing a device, comprising:
a substrate,
a first contact attached to the substrate,
a second contact attached to the substrate,
a first polynucleotide attached to the first contact and having a binding region, and
a second polynucleotide attached to the second contact and having a binding region;
contacting the device with one or more polynucleotide sequences, each with a first and second region of one or more unpaired bases;
introducing an electrical current to the device; and
testing for conduction of the electrical current between the first and second contacts,
wherein electrical conduction detected between the first and second contacts indicates hybridization of one of the one or more polynucleotide sequences to each of the first and second polynucleotides, and a lack of electrical conduction detected between the first and second contacts indicates a lack of hybridization of a polynucleotide sequence to each of the first and second polynucleotides.

26. The method of claim 25, wherein the testing further comprises employing one or more on-chip and/or external devices that are attached to the device.

27. The method of claim 26, wherein one of the one or more on-chip and/or external devices is an ammeter and/or a voltmeter.

28. The method of claim 25, wherein the device is configured to measure conductance and/or capacitance.

29. The method of claim 25, wherein the device further comprises one or more additional contacts attached to the substrate, and one or more additional polynucleotides each attached to one or more of the one or more additional contacts and each having a binding region, wherein either all binding regions are identical or all binding regions are not identical, and wherein testing for conduction of the electrical current further comprises testing for conduction of the electrical current between any pair of contacts included among the first contact, the second contact and the one or more additional contacts.

30. A method for determining single molecule kinetics, comprising:
providing a wiring system comprising:
a substrate,
a first electrode attached to the substrate,
a second electrode attached to the substrate, and
a polynucleotide attached to the first electrode;
applying a positive bias to the second electrode; and
monitoring the time elapsed between applying the positive bias to the second electrode and the attachment of the polynucleotide to the second electrode to determine single molecule kinetics.

31. A method for determining and analyzing the electrical properties of a polynucleotide sequences, comprising:
providing a device, comprising:
a substrate,
a first contact attached to the substrate,
a second contact attached to the substrate,
a first polynucleotide attached to the first contact and having a binding region, and
a second polynucleotide attached to the second contact and having a binding region;
contacting the device with a bridging polynucleotide sequence that hybridizes to the binding regions of each of the first and second polynucleotides; and
introducing an electrical current to the device; and
measuring the electrical properties of the bridging polynucleotide using a device selected from the group consisting of: semiconductor parameter analyzers, voltmeters, ammeters, pulse generators, potentiostats, galvanostats, function generators and combinations thereof, and wherein the measured electrical properties are selected from the group consisting of: conductance, capacitance, inductance and combinations thereof.
